(19) United States
(12) Patent Application Publication
(10) Pub. No.: US 2024/0194357 A1
(43) Pub. Date: Jun. 13, 2024

(54) SYSTEM AND METHOD TO AUTONOMOUSLY AND REMOTELY MONITOR, ANALYZE AND RESPOND TO PHYSIOLOGICAL AND DIAGNOSTIC DEVICE DATA FROM A MEDICAL IMPLANT

(71) Applicant: BIOTRONIK SE & Co. KG, Berlin (DE)

(72) Inventors: James Horton, Portland, OR (US); Lauren Kraiter, Tigard, OR (US); Andrew B. Kibler, Lake Oswego, OR (US); Andreas Gute, Portland, OR (US); Adrian Kimber, Glenview, IL (US); Brian Hills, Barrington, IL (US); Warren Dabney, Lake Oswego, OR (US); Sean Slee, Tigard, OR (US)

(21) Appl. No.: 18/286,817

(22) PCT Filed: Apr. 7, 2022
(86) PCT No.: PCT/EP2022/059244

§ 371 Date: Oct. 13, 2023

Related U.S. Application Data
(60) Provisional application No. 63/175,598, filed on Apr. 16, 2021.

(30) Foreign Application Priority Data
May 11, 2021 (EP) 21173347.2

(51) Int. Cl. G16H 80/00; G16H 40/67

(52) U.S. Cl. CPC G16H 80/00; G16H 40/67

(57) ABSTRACT

The present invention relates to a system for remote patient monitoring. The system comprises at least one medical device, a patient remote device, a remote monitoring server device (RMS), and a health care professional (HCP) remote device. The at least one medical device is configured to automatically collect physiological data of a patient and transmit the collected physiological data of the patient to the RMS via the patient remote device. The RMS is configured to interface with the HCP remote device via a healthcare professional interface comprising a web-based portal and/or an app-based portal to allow a health care professional to access the collected physiological data of the patient on the RMS. In this way, it is possible to provide continuous remote monitoring of physiological data from a medical implant with no patient interaction required. The monitoring data is available to physicians via web-based and/or app-based user portal on-demand. The RMS may provide frequent (e.g. daily) automatic reporting of physiologic measurements e.g. sleep impairment, activity levels, core temperature for fever (e.g. potential infections).

This disclosure describes a system and a method for remote patient monitoring.

State-of-the-art implanted pacemakers, cardiac monitors, neuro-stimulators and similar Implanted Medical Devices (IMD) communicate with clinician user interface (CUI) devices (“programmers”) during a face-to-face visit with a clinician. During the face-to-face visit the programmer provides a real-time view of diagnostic and technical data.

Data capture capability in the typical systems are lacking. For example, assessment of Spinal Cord Stimulation (SCS) patient status for pain management is presently limited to qualitative, subjective feedback from the patient and/or interrogated data gathered during an in-clinic follow-up. These processes typically lack objective assessment tools for review of current, consolidated patient and device status or the review of patient longitudinal data.

Many of the newer state-of-the-art implanted device systems can also transmit diagnostic and technical data periodically to a server (e.g. a “Remote Monitoring Server”, RMS). Clinicians access this data through a secure website or mobile apps. This enables them to remotely view their patients' status.

However, the User Interface (UI) provided for the clinicians to interact with this data typically provides no other service than a display of the patient IMD interrogation report collected in-office and/or via remote monitoring. The reports available are frequently too limiting for clinicians to provide more complex care to their patients or for use of this data in other aspects of the clinical practice management.

Moreover, the data transmission delays in existing remote monitoring designs are too limiting for clinicians to provide more complex care to their patients. For example, neurostimulation systems require interactive feedback from patient to clinician in order to adjust the stimulation therapy effectively. Providing such interactions remotely is not possible with today's systems. In Cardiac Rhythm Management (CRM), it is desirable to be able to view an Intracardiac Electrogram (IEGM) and device status in real-time when a patient contacts the clinic and reports the onset of an event. Viewing the IEGM and system data in real-time allows the clinician to accurately triage the patient and determine required next steps. Even though CRM devices are typically capable of detecting and recording events, it should not be assumed that the device has always been correctly programmed to detect the event. Further, as these implanted devices typically transmit based only upon an internal trigger (e.g. event detection, scheduled update transmission), the data transmitted from them is typically not current at the time of an in-office check. Accordingly, the device is typically interrogated at the start of any office visit, with resulting clinical labor cost and time delay. This function could be performed in advance of the patient visit if interrogation is available to the clinic remotely.

The present disclosure provides a system, a method, a computer program product and a computer readable data carrier for remote patient monitoring according to the independent claims, which overcomes the drawbacks of the prior art. Further embodiments are incorporated in the dependent claims.

According to a first aspect of the present disclosure, there is provided a system for remote patient monitoring. The system comprises at least one medical device, a patient remote device, a remote monitoring server device (RMS), and a health care professional (HCP) remote device. The at least one medical device is configured to automatically collect physiological data of a patient and transmit the collected physiological data of the patient to the RMS via the patient remote device. The RMS is configured to interface with the HCP remote device via a healthcare professional interface comprising a web-based portal and/or an app-based portal to allow a health care professional to access the collected physiological data of the patient on the RMS.

The proposed system may have the advantage that it is possible to provide continuous remote monitoring of physiological data from a medical implant with no patient interaction required. The monitoring data is available to physicians via web-based and/or app-based user portal on-demand. The RMS may provide frequent (e.g. daily) automatic reporting of physiologic measurements e.g. sleep impairment, activity levels, core temperature for fever (e.g. potential infections). Monitoring capability may be turned on via the healthcare professional interface, the manufacturer support interface, and/or the patient interface as described below.

The proposed system may be used in multiple areas of medical device application, e.g. spinal cord stimulation (SCS), cardiac rhythm management (CRM), and many other medical fields that utilize an implanted medical device e. g. deep brain stimulation (DBS), occipital nerve stimulation (ONS), trigeminal nerve stimulation (TNS), vagal nerve stimulation (VNS), phrenic nerve stimulation, gastric electrical stimulation, and sacral nerve stimulation (SNS).

According to an embodiment of the present invention, the system is configured to provide real-time remote programming and/or interrogation of the at least one medical device using the healthcare professional interface via the patient remote device.

In this way, clinicians do not rely on the patient to initiate the data capture and data transmission, and are therefore in control of how or when the data is captured. As the clinic can control receipt of the information transmitted from the implant, they are able to respond to the receipt of the data at the time it is received. This may lead to an improved workflow regarding the clinic review of the data.

Further, it is possible for clinical expert (e.g. company service personnel) to remotely connect, confirm implant device status, and send new or updated therapy program(s) to the patient. The workflow can be customized for each physician and patient (e.g. option to participate or not in remote patient visit).

According to an embodiment of the present invention, the patient remote device is further configured to interface with RMS via a patient interface comprising a web-based portal and/or an app-based portal to (i) allow the patient to provide patient-provided data and transmit the patient-provided data to the RMS, and/or (ii) to allow the patient and/or a patient caregiver to access a subset of patient-appropriate data for the patient.

In this way, it is possible to create patient-reported symptom data, via the patient remote device (e.g. smart phone), to coordinate early and effective treatment.

In addition, it is known within the field of spinal cord stimulation that the more personalized attention that is given to the patient, the greater the chances of success and patient satisfaction with the stimulation. The introduction of remote interaction with the patient-provided data (even without advanced analysis) may enable the clinician or company representative to provide more frequent and involved personalized attention without the clinician/rep or patient having to travel.

According to an embodiment of the present invention, the patient-provided data comprises one or more of:
patient-reported remote pain assessment;
patient-reported remote sleep assessment; and
patient-reported symptom data.

According to an embodiment of the present invention, the patient remote device is configured to provide interrogation of diagnostic device data of the at least one medical device and transmit the diagnostic device data to the RMS.

This may allow automatic routing of daily device diagnostics that can impact patient compliance (e.g. electrode impedance, lead shifts, stimulation usage, state of charge (e.g. battery status), therapy on/off).

According to an embodiment of the present invention, the RMS is configured to interface with a manufacturer support via a manufacturer support interface comprising a web-based portal and/or an app-based portal to allow the manufacturer support to access the collected physiological data of the patient and/or the diagnostic device data of the at least one medical device.

According to an embodiment of the present invention, the RMS is configured to monitor the collected physiological data of the patient and/or the patient-provided data and to generate an alert indicative of a likely change in the patient's health or mental well-being upon an occurrence of one or more pre-set triggers. Alternatively, or additionally, the RMS is configured to monitor the diagnostic device data and to generate an alert indicative of an impacted patient compliance, upon an occurrence of one or more pre-set triggers.

In this way, it is possible for proactive outreach by monitoring personnel to the patient/HCP when remote monitoring identifies an opportunity to optimize therapy. For example, the alert may trigger a need for a certain action to be taken by remote management (i.e. reprogramming, clinical visit, and/or referral) to optimize therapy.

According to an embodiment of the present invention, the one or more pre-set triggers are configurable via the healthcare professional interface and/or the manufacturer support interface.

According to an embodiment of the present invention, the one or more pre-set triggers are configurable on a per patient basis or a per patient group basis. Alternatively, or additionally, the one or more pre-set triggers are configurable for a destination of the generated alert.

According to an embodiment of the present invention, the RMS is configured to generate the alert with a level of priority.

For example, alerts could be categorized as high priority or normal priority as part of the configuration set-up. These alert priorities, for an individual alert recipient or recipient group, may control if and how a notification is received and/or the organization and appearance of the alerts on the front-end interfaces.

According to an embodiment of the present invention, the one or more pre-set triggers are initially configurable via the healthcare professional interface. The RMS is configured to autonomously and/or continuously update the one or more pre-set triggers based on a need of the patient.

For example, the autonomous triggers may be initially set by the clinician, but then autonomously monitored and updated by an artificial intelligence (AI) process to further fine-tune the process for the needs of the individual patient.

According to an embodiment of the present invention, the RMS is configured to generate a ticket upon an occurrence of one or more pre-set triggers and to share the ticket amongst a plurality of manufacturer support users via the manufacturer support interface.

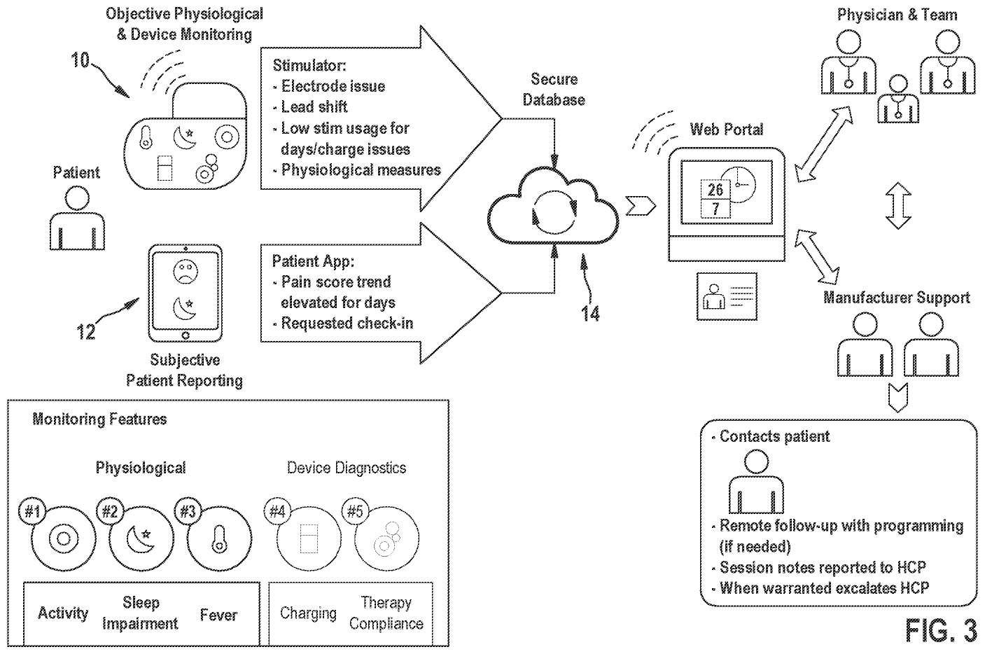

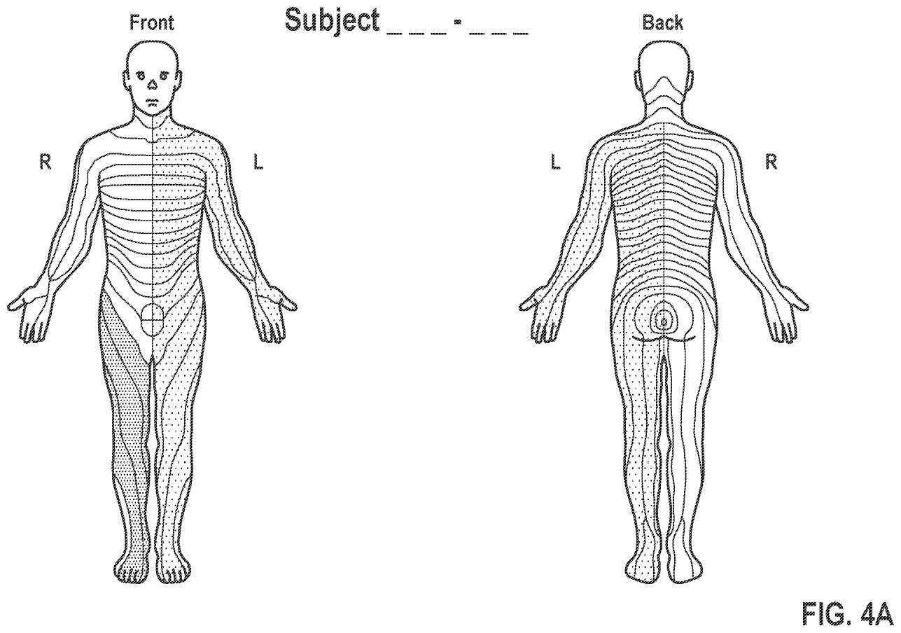

The ticketing system will be described hereinafter and in particular with respect to the embodiment shown in FIG. 3.

According to a second aspect of the present disclosure, a method for remote patient monitoring is provided. The method comprises:
automatically collecting, by at least one medical device, physiological data of a patient;
transmitting, by the at least one medical device, the collected physiological data of the patient to a remote monitoring server device, RMS, via a patient remote device, and;
interfacing, by the RMS, with a healthcare professional, HCP, remote device via a healthcare professional interface comprising a web-based portal and/or an app-based portal to allow a health care professional to access the collected physiological data of the patient on the RMS.

According to a third aspect of the present disclosure, there is provided a computer program product comprising instructions, which, when executed by at least one processing unit, cause the at least one processing unit to perform the steps of the method according to the second aspect.

According to another aspect of the invention, there is provided a computer readable data carrier storing a computer program product.

The invention will be described in detail with reference to the following figures:

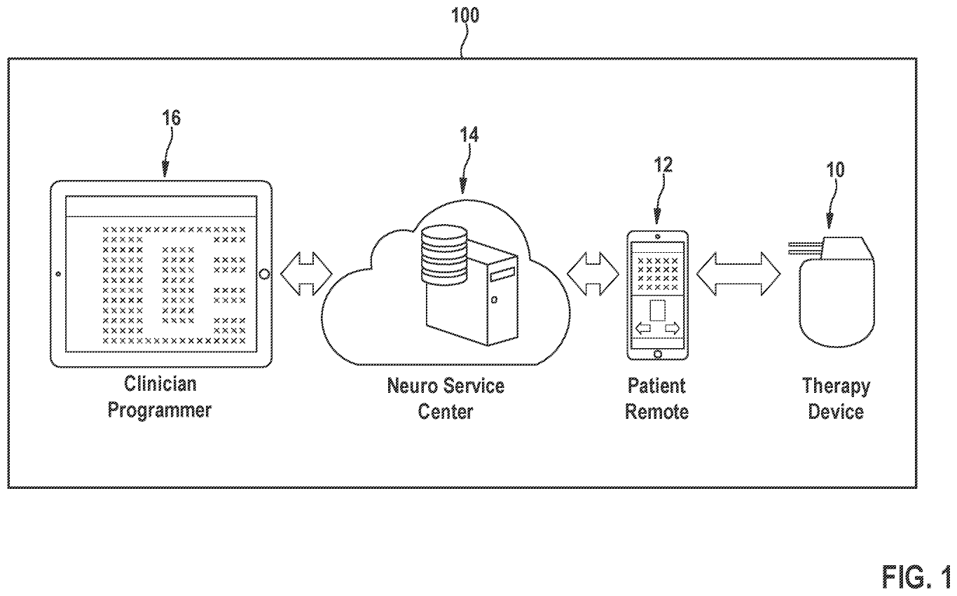

FIG. 1 illustrates components of a system for remote patient monitoring.

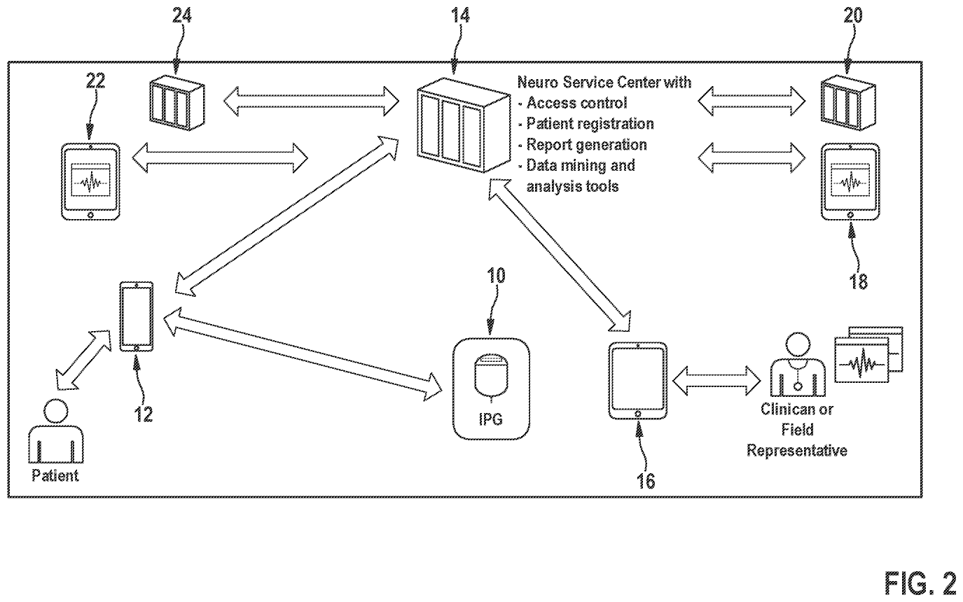

FIG. 2 illustrates a system architecture of the described system for remote monitoring and remote programming.

FIG. 3 illustrates an example of a workflow of the described system for remote monitoring and remote programming.

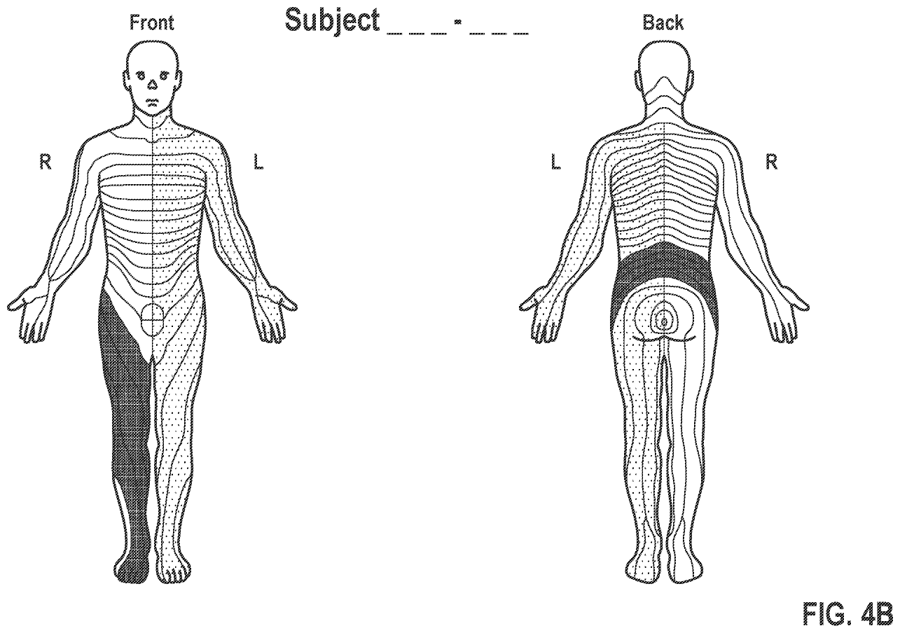

FIG. 4A illustrates that a notification is sent to the patient remote device to complete a new pain map.

FIG. 4B illustrates a patient selected section in lower back corresponding to the current pain.

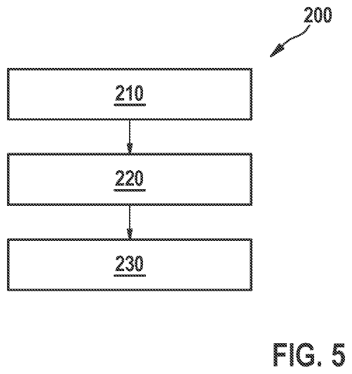

FIG. 5 illustrates a flow chart for a method for remote patient monitoring.

In the following, the invention will be described in detail by means of the aforementioned figures. The exemplary embodiments illustrated therein serve for illustrative purposes, wherein the matter of the invention is not limited by the exemplary embodiments, but defined by the subject matter of the claims.

In the following, embodiments of the inventive system will be explained in detail. The embodiments are explained with regard to medical devices in the form of spinal cord stimulation (SCS) devices and a remote monitoring server (RMS) in the form of a Neuro Service Center (NCS) and with regard to remote programming which may include data interrogation.

FIG. 1 shows components of the system 100. The system 100 comprises at least one medical device 10, a patient remote device 12, a remote monitoring server device (RMS) 14, and a health care professional (HCP) remote device 16.

The at least one medical device 10 may be referred to as “therapy device”, which may be e.g. an implanted medical device (e.g. implantable monitoring device/loop recorder, trial stimulator, stimulator or other implantable pulse generator). The at least one medical device 10 is configured to automatically collect physiological data of a patient and transmit the collected physiological data of the patient to the RMS via the patient remote device. Examples of the collected physiological data of the patient may include, but are not limited to, sleep impairment, activity levels, core temperature for fever, etc.

The patient remote device 12 serves as a transceiver to route communication between the at least one medical device 10 and the RMS 14. The patient remote device 12 may provide the patient some aspect of local control of the therapy process. For example, it may give the patient the ability to change the active therapy program of a medical device, for example, a medical device for spinal cord stimulation (SCS), control its stimulation amplitude, turn stimulation on/off, and view battery status. It may also serve as an external programmer for the medical device.

The RMS 14 is, for example, a hardware and/or cloud-based service center that facilitates Remote Programming (RP) and data interrogation of the at least one medical device, and may comprise a central data repository of data collected by the at least one medical device. In the example of FIG. 1, the RMS is in the form of a Neuro Service Center (NRS). The hardware may comprise at least one computing device or a network of a plurality of computing devices. The RMS further comprises the computing functionality described herein. For example, the RMS is a functional unit that performs substantial computations; including numerous arithmetic operations and logic operations without human intervention and may comprise, such as, for example, a desktop computer: a server computer, clusters/warehouse scale computer or embedded system.

The HCP remote device 16 may also be referred to as clinician programmer.

The HCP remote device may be used by healthcare professionals or field technical representatives to program the medical device remotely. The RMS 14 is configured to interface with the HCP remote device 16 via a healthcare professional interface comprising a web-based portal and/or an app-based portal to allow a health care professional to access the collected physiological data of the patient on the RMS. The HCP remote device 16 may be physical (i.e. dedicated device) or virtual (e.g. via web-based interface to the system, secure connected data reporters).

FIG. 2 illustrates a system architecture of the described system 100 for remote monitoring and/or remote programming.

For the SCS embodiment, the healthcare professional interface (also referred to as NSC User Interface) on the HCP remote device 16, may enable a closed loop, real-time communications with the at least one medical device 10. For example, clinician wants to know patient status in preparation for a follow-up consult or remote assistance call with the patient. Clinician triggers an “update data” request from the RMS, which is transmitted to the patient remote device 12 (e.g. a mobile phone or a wearable like a smart watch), which in turn relays the request to the at least one medical device 10, e.g. implant pulse generator (IPG) shown in FIG. 2. The implant queries current patient and device status, and transmits a report to the RMS 14 via the patient remote device 12. The report may be then received and processed (i.e. the message is converted to a form usable by the RMS, scanned for alert conditions using system algorithms, and assigned to the correct clinic and patient) by the RMS. At this point, the clinician may be able to review the new report, in advance of the in-person meeting.

In some examples, the RMS may be configured to combine subjective data (e.g. patient diary, patient surveys, clinician notes, etc.) and objective data (physiological and/or technical data such as current patient status, patient trends and outcomes analysis, activity-based tracking, medication use tracking, longitudinal pain data, statistics and therapy data such as eCap) into one viewable database. Data to be combined may be retrieved from multiple data reporters, such as HCP remote device, patient remote device, RMS, medical devices and others.

In some examples, the RMS may provide a real-time website display of consolidated data accessible by multiple users. For example, the healthcare professional interface (e.g. clinician's web interface) may be used to display:
Data received via regular routine to collect data automatically (daily, monthly, etc.) or upon request (e.g. preparation for in office visit, patient adjustment, etc.);
A current patient status “snapshot” collected in near real-time showing all quantitative and subjective data stored for that patient: online access to patient remote device and/or the at least one medical device to collect real-time data;
The status of the patient's system and statistics collected over time (e.g. implanted lead integrity, present stimulation settings, and diagnostic trends related to pain, and/or system use);
The system displays current program parameters in use by the patient device, and;
Optionally, the user may have the ability to configure the display of the combined data such that different data trends are added or removed from the display according to the user's preferences.

In some examples, the healthcare professional interface may be expanded to accept and display other data types, such as patient image, video, and/or audio files, and remote assessment of neurostimulation for pain.

In some examples, the RMS may be configured to enable patient database access control. In an example, the RMS may be configured to enable controlled device access by providing a means of registering Clinician Programmer users and controlling their access to remote follow-up. In another example, the RMS may be configured to enable configurable user access rights such that data access to individual Patient Health Record (PHR) is controlled on a per-user basis.

In some examples, the RMS may be configured to enable patient database connectivity. For example, NSC database may be accessible via multiple portals such as healthcare professional interface, patient interface, manufacturer support interface, and others.

For example, internal Data Consumers 18 and Data Reporters 20 within the system 100 may read/write to the database via system-supplied user interfaces. E.g. Technical Services has the ability to review raw patient data and to write to the patient database.

Optionally, the RMS may allow system users to both pull data from the RMS 14 and push data from the patient remote device to the RMS 14. For example, an external data consumer 22 such as an Electronic Health Record (EHR) system may securely query and pull patient data from the RMS based upon its own internal schedule, or on-demand during a real-time session. For example, the RMS 14 may export pre-selected data to an external data consumer 22, based upon a schedule previously configured by the user. Optionally, external data consumers 22 and data reporters 24 (e.g. EHR system) may connect to the RMS via system-supplied Application Programming Interfaces (API).

In some examples, the RMS 14 may be configured to enable queries to a searchable database of all patients and devices under the control of that clinic.

In some examples, the RMS 14 may be configured to enable configurable report formats by allowing customization to clinic or individual user needs.

In some examples, the RMS 14 may be configured to allow “Electronic Signature” or “Digital Signature” capability to allow the physician and/or patient to electronically provide consent for future participation in Remote Follow-up.

In some examples, the RMS 14 may be configured to allow capability for viewing population-wide data for the clinic or for a device registry, such as looking at overall outcomes or relationships of patient characteristics.

FIG. 3 illustrates an example of a workflow of the described system for remote monitoring and remote programming.

In general, when patient information is created, the patient may be assigned to one or more patient groups (such as clinic group or field support region). These groups may be changed later, as needed, via system admin user interface.

For each individual patient, agreement must be documented before monitoring capability is turned on. Optionally, the patient programmer or patient app may include the agreement.

Monitoring capability could be turned on via the manufacturer support interface, the healthcare professional interface, and/or the patient interface. Users of the monitoring data must have individual accounts created with which patient groups the user is allowed to access and, optionally, preferences for alert notifications, including contact information if applicable.

### Alerts

In some examples, the RMS 14 is configured to monitor the collected physiological data of the patient and/or the patient-provided data and to generate an alert indicative of a likely change in the patient's health or mental wellbeing upon an occurrence of one or more pre-set triggers. Alternatively or additionally, the RMS 14 is configured to monitor the diagnostic device data and to generate an alert indicative of an impacted patient compliance upon an occurrence of one or more pre-set triggers.

For example, the alert that is indicative of a likely change in the patient's health or mental wellbeing could include one or more of the following:
Minutes of activity per day (for Y days moving average) has dropped below or risen above X;
Minutes of resting positions per day (for Y days average) has dropped below or risen above X
Heart rate per minute (for Y days average) has dropped below or risen above X
Sleep restlessness score (according to accelerometer motion algorithm, for Y days moving average) has dropped below or risen above X;
Body temperature elevated above X, indicating a suspected fever or infection;
Patient-reported pain score elevated above X for Y days;
Patient-reported sleep score dropped below X for Y days;
Additionally, any combination of conditions, such as both patient-reported pain and accelerometer-measured activity reaching a threshold suggesting possibility of worsening patient condition;
Patient data not reviewed for X days; and
Regularly scheduled remote follow-up is due according to fixed date or interval schedule.

For example, the alert that is indicative of an impacted patient compliance could include one or more of:
Lead impedance out-of-range on a therapeutic electrode;
Suspected lead shift greater than X contacts has occurred;
Stimulation usage has been below X % for the last Y days;
Battery has depleted, resulting in automatic shut-off of stimulation;
MRI mode is left on for X days;
Active therapy program has been changed from the preferred therapy program; and
Active therapy program amplitude has dropped below X for more than Y days.

In another embodiment, the system would provide “active” patient alerts. For example, the system provides a “push” alert, generated autonomously within the system, as compared to only generating a passive/“pull” display of data in the user interface of the patient remote device, or the HCR remote device. In one embodiment, the patient remote device communicates data to the RMS that stimulator battery charge is low as per requirements to safely deliver therapy (the trigger settings for determination that alert conditions have been met can reside in either the RMS or in the patient remote device itself). The system subsequently sends a message to the patient's personal phone (e.g. via a patient app) informing the patient of the low charge condition.

Moreover, according to another embodiment, if the system detects certain complications during charging of the medical device, the system automatically sends educational information to the patient app that could guide the patient through the best charging process (e.g. “Charging education”). The complications during charging may be determined from observed behavior as frequently charging stopped before charge is complete, or excessive time between recharge sessions, or other use patterns that suggest that patient is not charging the battery in a manner consistent with best practices.

In another embodiment, the system would provide an estimated battery use for a program associated with the medical device. For a therapy program available on the medical device, the system computes an estimate on the running time until recharging is needed. This info could be available as both “pull” data, viewable on demand by the user, or as a “push” alert when current program use could lead to rapid depletion of the battery. Such info could be available to both the HCP and patient remote device, or the patient personal device (e.g. mobile phone). In this embodiment the RMS would calculate the estimated battery use instead of putting the calculation on the HCP remote device, which may be only intermittently connected. According to an embodiment, the estimated battery use is computed for an actively running therapy program of the medical device.

In another embodiment, the system would provide patient automatic therapy recommendations. For instance, data obtained by or associated with the medical device and monitored via the RMS are used as a trigger for automatic recommendations sent to the patient remote device. This recommendations system is for instance programmed according to a decision tree. As an example, if the medical device is a neurostimulator for treating pain, including monitoring a patient's pain score, the system can autonomously suggest to the patient to try a new default program if a patient's pain score is still elevated 5 days after a reprogramming session. The decision tree used could use patient specific characteristics, or it could be purely general recommendations based on a set decision tree.

In some examples, alerts may be configured through the front-end user interface. For example, the physician/clinic team may define a list of alerts and triggering conditions.

In some examples, separate alerts may be configurable for the manufacturer support team and physician/clinic team. Optionally, individual users may be defined within the physician/clinic team to have individually customized alerts. For example, the physician is only notified of temperature change, but the nurse is notified of all alerts. Optionally, the manufacturer team may access and change the physician/clinic users' alerts as a means of aiding those users. Optionally, within the manufacturer team, individual users may be defined to have individually customized alerts. For example, the local field rep is only notified of certain alerts, but the in-house customer support team receives all alerts.

In some examples, alerts may be customized on a per patient basis. In other words, individual users may be defined to have individually customized alerts.

In some examples, alerts may be customized on a per-patient group basis. For example, the clinical users may have the option to set consistent alerts across all patients associated with the clinic. For example, the manufacturer users have the option to set consistent alerts across all patients within a clinic group.

In some examples, also configurable may be where notifications are sent for the alerts. For example, each clinical user may choose from input contact information such as SMS or email for sending notifications or choose to have notifications present on the web interface only. Optionally, the notifications may be integrated with the clinic's electronic health record system.

Optionally, alerts could be categorized as high priority or normal priority as part of the configuration set-up. These alert priorities, for an individual alert recipient or recipient group, may control e.g. if and how a notification is received.

Optionally, the autonomous triggers can be initially set by the clinician, but then autonomously monitored and updated by an AI process to further fine-tune the process for the needs of the individual patient.

Portal for Manufacturer Support Team, i.e. Manufacturer Support Interface

Central to the function of the manufacturer support team interface may be a ticketing system, based on alerts. This ticketing system may be shared amongst manufacturer support users.

In some examples, new “tickets” generated by alert conditions may be assigned to an individual manufacturer support user. Each individual manufacturer support user may have a unique queue of tickets for viewing and addressing, ordered by priority of alerts and/or time since alert was detected and/or priority of clinical accounts.

In some examples, each ticket may be linked to an individual patient master record that comprises monitoring data (e.g. collected physiological data, patient-reported data, and/or diagnostic device data) for the patient, past tickets and associated notes with resolving the ticket, and/or any related clinic correspondence.

In some examples, the ticketing system may also allow tickets to be added manually, for example, if a patient or clinician calls the support center directly.

In some examples, the ticketing system may allow for statuses to be assigned to the tickets, including e.g. assess, reach out to patient, report to physician, follow-up, etc. The ticketing system may enable the support users to consistently process tickets of the same type by providing specific prompts with selectable drop-down menu selections to complete. For example, a ticket in the “needs assessment” state could include a drop-down to respond to “assessment result” with options for “refer to physician”, “reach out to patient for more information”, or “no intervention needed”.

The ticketing system may track historical records of the time, involved users, and/or added data/notes for each modification of the tickets.

In some examples, the manufacturer support team portal may also include the ability to view monitoring data regardless of whether it is tied to alerts. The format of the monitoring data may include one or more of the following:
Ability to view a historical list and associated notes with key events for the patient, such as in-office follow-ups, remote follow-ups, and other interactions;
Ability to view a snapshot of the most recent measurements and the date on which those measurements were taken;
Ability to view trend plots of the data with flexible trend duration views. Optionally, trend plots could be customizable to include more than one variable on the same trend. For example, the user may choose to have activity and sleep quality appear on the same graph. In addition to supporting varying duration views, trends plots may support providing detail on individual data points (e.g. dates and specific values) of interest. Trend plots may also be modified to visually include key events, such as in-office follow-ups, remote follow-ups, and other interactions with the patient;
Ability to view tabular comparisons at two or more specific time points, for example, pre-implant baseline values on a single day or specified X-day average pre-implant verses the most recent X day average; and
Ability to generate customized reports for sending to the physician/clinic via the healthcare professional interface (e.g. the clinic web portal). Customized report options may be selectable for which monitoring data is included, what duration of monitoring data is included, specific notes from the ticketing system to be included, and additional customizable notes to the physician office.
   Portal for the Physician/Clinic, i.e. Healthcare Professional Interface

The web-based or app-based portal for the physician/clinic could include all functionality described in the manufacturer's portal, or a subset of that functionality. In general, in addition to the possibilities described above, the clinic portal may also include one or more of the following:
Ability to define default view for the clinic, including which data are displayed, order of displayed data, and duration of each type of data;
Ability to define a list of predefined reports for the individual patient, for which the clinic user is able to quickly select and export (such as for use in the EHR for remote monitoring billing);
Ability to view a summary of all of the clinic's patients' key outcomes data as defined by clinic preferences (for example, a listing of all patients by implant date, within 3, 6, 12, and 24 months, and most recent pain score and activity values). Within this summary, individual patients may be selectable for drilling down to more detailed patient information; and
Ability to track the amount of time the user spends within the system viewing an individual patient e.g. for remote monitoring billing purposes.
   Portal for the Patient and/or Caregiver(s)

A subset of data could be selected for display in a patient-friendly format on a patient and/or caregiver web portal or app on the patient device 12. The subset of data could be defined according to selections made by the clinic, by a member of the manufacturer support team, or by the patient or caregiver user him/herself.

A specific aspect of the present disclosure is that a set of assessments that may be utilized in combination with the remote management to ensure effective daily pain relief and high quality of life over the lifetime of the implanted device. These assessments may be triggered either autonomously, based upon pre-set criteria, or manually by the clinician or patient.

In general, these assessments may trigger an action from remote management individually or based on a combination of responses from different assessments. Data science approaches may be utilized to determine which assessment responses or groups of responses are most predictive of a need for a certain action to be taken by remote management (e.g. reprogramming, clinical visit, referral). These approaches may include e.g. artificial neural networks, linear and logistic regression, clustering and dimensionality reduction among others.

For example, the technical process of the assessment triggering an actionable output may be summarized as follows:
1. Patient completes assessment and upload results to NSC (cloud database);
2. Custom software reads in the data and converts the assessment results into input variables (can be alpha-numeric or graphical data);
3. Software compares the input variables (or quantities computed from the input variables) to a predetermined threshold value; and

4. If the input exceeds the threshold the custom software creates and transmits an alert.

Patient-reported remote pain assessment

In some examples, remote management system or components, such as the RMS 14, may send the patient a notification via their patient remote devices 12 to complete a chronic pain assessment. This could be performed on a pre-determined schedule as part of a clinical study, by request of the patient, or by request of a physician, possibly prior to a clinical follow-up visit.

An example of the remote pain assessment is described in the following:

Step 1: Notification is sent to the patient remote devices. Examples of the notification may be e.g.:
“How much BACK pain are you experiencing now, from 0 (No Pain) to 10 (Worst Possible)?”
“How much LEG pain are you experiencing now, from 0 (No Pain) to 10 (Worst Possible)?”
“How much OVERALL pain are you experiencing now, from 0 (No Pain) to 10 (Worst Possible)?”

Step 2: Patient completes assessment. In response to each question, the patient may either enter a number or move a slider to indicate their current pain score.

Step 3: Patient submits survey through secure cloud-based remote management, which may trigger one or more of the following potential actions:
Consistent increase in pain score may trigger a follow-up remote or in-person visit. For example, a threshold may be defined (e.g. Numerical Rating Scale (NRS)>3 points above daily NRS averaged over the preceding month on three consecutive days). An alert (e.g. text message or email) may be sent to the clinician regarding pain increase. The message may contain a link to schedule a follow-up with the patient (that way NSC knows action has been taken); alternatively an AI automatically analyses and proposes to the patient and/or the physician appropriate adjustments taking in account the increase in pain score;
Consistent decrease may trigger an adjustment of parameters (perhaps lower energy leading to longer recharge interval). Similar type of trigger may prompt a clinician alert: alternatively an AI automatically analyses and proposes to the patient and/or the physician appropriate adjustments taking in account the decrease in pain score.
During trial, high pain score may trigger a remote programming session to send a new test therapy. Similar type of trigger prompting a clinician alert, lower threshold here e.g. triggered by smaller increase in NRS score due to time constraints of the trial

### Patient Reported-Remote Sleep Assessment

In some examples, remote management may send the patient a notification via their patient remote devices 12 to complete a sleep quality assessment. This could be performed on a pre-determined schedule as part of clinical study, by request of the patient or by request of a physician, possibly prior to a clinical follow-up visit. The assessment interval could be e.g. last night, over the last week, or month since therapy optimization.

An example of the remote sleep assessment is described in the following:

Step 1: Notification is sent to the patient remote devices. Examples of the notification may be e.g.: “How well did you sleep last night, from 0 (Worst Possible) to 10 (Best Possible)?”

Step 2: Patient completes assessment. The patient may either enter a number or move a slider to indicate last night's sleep score.

Step 3: Patient submits survey through secure cloud-based remote management, which may trigger one or more of the following potential actions:
Consistent decrease in sleep score (poor sleep) triggers a follow-up remote or in-person visit or another assessment to determine if it is pain-related. For example, a threshold may be defined (e.g. sleep score averaged over one week less than 3 points below sleep score averaged over the preceding month). An alert (e.g. text message or email) may then be sent to the clinician regarding a change in sleep score. The message may contain a link to send the patient an additional survey to assess if the poor sleep is the result of an increase in pain); alternatively an AI automatically analyses and proposes to the patient and/or the physician adjustments taking in account the decrease in sleep score;
Result of this or other assessments may trigger a follow-up remote or in-person visit: and
During trial, it may trigger a remote programming session to send a new test therapy or alert the clinician to assist with interpretation of subsequent pain score since sleep quality and pain are correlated.

### Patient Reported-Remote Pain Map

In some examples, remote management may send the patient a notification via their patient remote devices to complete a chronic pain map. This could be performed on a pre-determined schedule as part of clinical study, by request of the patient or by request of a physician, possibly prior to a clinical follow-up visit. Ideally, the patient will have completed an initial pain map at the beginning of the trial, permanent implant or both.

An example of the patient reported-remote pain map is described in the following:

Step 1: Patient completes a baseline chronic pain map e.g. prior to trial or permanent implant. For example, the patients may select regions on pain map where they experience chronic pain. This can be accomplished using standard graphical software and a touch screen such as that available on the healthcare professional remote device.

For example, FIG. 4A shows that a notification is sent to the patient remote device to complete a new pain map. The map contains shading to indicate their previously reported baseline pain.

Step 2: Patient completes pain map assessment by selecting sections corresponding to their current pain

For example, FIG. 4B shows a patient selected section in lower back and front leg corresponding to the current pain.

Step 3: Patient submits current pain through secure cloud-based remote management, which may trigger one or more of the following potential actions:
Increase in pain area may trigger a follow-up remote or in-person visit. For example, new back pain may lead to reprogramming by shifting active contacts superior; reprogramming can be done by AI or support of AI
Decrease in pain area may trigger an adjustment of parameters (perhaps lower energy leading to longer recharge interval). Alert (e.g. text or email) may be sent to clinician notifying them of a reduction in pain area. The alert contains a link to schedule a follow-up with the patient: alternatively an AI automatically analyses and proposes to the patient and/or the physician adjustments taking in account the decrease in pain area; and
During trial, triggers a remote programming session to send a new test therapy.
   Ability to Remotely Monitor Physiological and Diagnostic Device Data from the Implant (w/No Patient Interaction Required):

### Monitoring of Daily Activity Levels

Daily activity levels may be calculated from a motion/acceleration detection circuit built into an implantable device for pain management. For example, the motion detection circuit may integrate counts representing activity (e.g. Accelerometer counts sampled at 8 Hz) in a short epoch (E1) for example, one second in duration. At the end of E1, the accumulated count value (Cv) may be compared against a multiplicity of threshold levels, for example L0-L3. A series of counters C0-C3 representing levels L0-L3 may be implemented, and the respective counter may be incremented depending on the corresponding threshold range Cv falls within.

In some examples, a longer epoch E2, for example 10 minutes, may be defined wherein the current values of C0-C3 may be saved to a storage record Rx and then C0-C3 are reset to zero at time periods of multiples of E2, resulting in activity records RO . . . Rx. For example, 144 records per day with E2 of 10 minutes.

The activity records may be stored in the implanted device and then transmitted to a central database of the RMS.

In some examples, the activity records stored in the database may be processed for display, representing a patient's activity at various intensity levels 10-13 corresponding to threshold levels L0-L3.

In some examples, the activity records stored in the database may be utilized as an objective performance metric in the assessment of patient wellbeing during their medical care.

In some examples, the activity records stored in the database may be utilized to evaluate activity goals set by the patient as a measure of improvement of their quality of life during medical care.

In some examples, the activity records stored in the database may be utilized to identify ‘high activity’ and ‘low activity’ periods of time whereby presence or absence of a desired count of respective periods may trigger an alert to a caregiver or device management assistant to follow up with the patient.

In some examples, the activity records stored in the database may be segmented into time periods within a 24-hour period representing at least one ‘wake’ period and at least one ‘sleep’ period, which is used for evaluating presence of expected periods of activity or improvements in intensity or duration of activity during the ‘wake’ period(s) and evaluating absence of activity during the ‘sleep’ period(s) and reporting a composite metric of sleep quality and efficiency derived from absence of activity during the ‘sleep’ period(s).

In some examples, the above described metrics and trends may be utilized to identify changes in activity behavior of patients compared to a baseline or compared to a historical trend, such that a substantial deviation away from the calculated baseline (e.g. historical average) or trend (linear or cyclic predictive model) triggers an alert to a caregiver (email, text, other notification system). The alert may represent a reliable behavior-derived indicator of a change in the patient's health or mental wellbeing. For example, the alert may represent a likely change in the pain state or ability to cope with pain of a pain patient. The alert may be used to trigger a patient care action or initiate an appointment.

### Temperature Monitoring

The at least one medical device may be configured to house a temperature sensor, said temperature sensor collecting and relaying temperature data to a central server as previously described.

In some examples, the at least one medical device may possess the additional ability to identify periods of time whereby its internal temperature reading is not representative of a patient's body temperature, e.g. during charging of the implantable device. Upon identification of these periods of time, the system may prevent the temperature readings corresponding to these periods of time from influencing a representative patient body temperature metric which is recorded and displayed to an end user (e.g. physician, etc.). Temperature measurements recorded in such a way may be used to track a baseline trend (average) and identify deviations from this baseline trend. These deviations may then generate an alert to a caregiver that the patient's body temperature surrounding the implanted device has deviated from the standard trend, potentially as a result of infection or other cause resulting in an altered metabolic state.

With reference to FIG. 5, a flow chart is shown for a method 200 for remote patient monitoring.

In step 210, at least one medical device automatically collects physiological data (e.g. sleep impairment, activity levels, and/or core temperature for fever) of a patient.

In step 220, the at least one medical device transmits the collected physiological data of the patient to a remote monitoring server device (RMS) via a patient remote device.

In step 230, the RMS interfaces with a healthcare professional (HCP) remote device via a healthcare professional interface comprising a web-based portal and/or an app-based portal to allow a health care professional to access the collected physiological data of the patient on the RMS.

In this way, it is possible to provide continuous remote monitoring of physiological data from a medical implant with no patient interaction required. The monitoring data is available to physicians via web-based and/or app-based user portal on-demand.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an update turns an existing program into a program that uses the invention.

Further on, the computer program element might be able to provide all necessary steps to fulfil the procedure of an exemplary embodiment of the method as described above.

According to a further exemplary embodiment of the present invention, a computer readable medium, such as a CD-ROM, is presented wherein the computer readable medium has a computer program element stored on it, which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

1. A system (100) for remote patient monitoring, comprising:
(a) at least one medical device (10);
(b) a patient remote device (12);
(c) a remote monitoring server device, RMS (14); and
(d) a health care professional, HCP, remote device (16);
wherein the at least one medical device is configured to automatically collect physiological data of a patient and transmit the collected physiological data of the patient to the RMS via the patient remote device; and
wherein the RMS is configured to interface with the HCP remote device via a healthcare professional interface comprising a web-based portal and/or an app-based portal to allow a health care professional to access the collected physiological data of the patient on the RMS.

2. The system according to claim 1,
wherein the system is configured to provide real-time remote programming and/or interrogation of the at least one medical device using the healthcare professional interface via the patient remote device.

3. The system according to claim 1,
wherein the patient remote device is further configured to interface with the RMS via a patient interface comprising a web-based portal and/or an app-based portal to;
(a) allow the patient to provide patient-provided data and transmit the patient-provided data to the RMS; and/
(a) allow the patient and/or a patient caregiver to access a subset of patient-appropriate data for the patient.

4. The system according to claim 3,
wherein the patient-provided data comprises one or more of:
(a) patient-reported remote pain assessment;
(b) patient-reported remote sleep assessment; and
(c) patient-reported symptom data.

5. The system according to claim 1,
wherein the patient remote device is configured to provide interrogation of diagnostic device data of the at least one medical device and transmit the diagnostic device data to the RMS.

6. The system according to claim 1,
wherein the RMS is configured to interface with a manufacturer support via a manufacturer support interface comprising a web-based portal and/or an app-based portal to allow the manufacturer support to access the collected physiological data of the patient and/or the diagnostic device data of the at least one medical device.

7. The system according to claim 1,
wherein the RMS is configured to:
(a) monitor the collected physiological data of the patient and/or the patient-provided data and to generate an alert indicative of a likely change in the patient's health or mental wellbeing upon an occurrence of one or more pre-set triggers; or
(b) monitor the diagnostic device data and to generate an alert indicative of a likely impacted patient compliance upon an occurrence of one or more pre-set triggers.

8. The system according to claim 7,
wherein the one or more pre-set triggers are configurable via the healthcare professional interface and/or the manufacturer support interface.

9. The system according to claim 8,
wherein the one or more pre-set triggers are configurable on a per patient basis or a per patient group basis; or
wherein the one or more pre-set triggers are configurable for a destination of the generated alert.

10. The system according to claim 7, wherein the RMS is configured to generate the alert with a level of priority.

11. The system according to claim 7, wherein the one or more pre-set triggers are initially configurable via the healthcare professional interface; and
wherein the RMS is configured to continuously update the one or more pre-set triggers based on a need of the patient.

12. The system according to claim 7, wherein the RMS is configured to generate a ticket upon an occurrence of one or more pre-set triggers and to share the ticket amongst a plurality of manufacturer support users via the manufacturer support interface.

13. A method (200) for remote patient monitoring, comprising:
(a) automatically collecting (210), by at least one medical device, physiological data of a patient;
(b) transmitting (220), by the at least one medical device, the collected physiological data of the patient to a remote monitoring server device, RMS, via a patient remote device; and
(c) interfacing (230), by the RMS, with a healthcare professional, HCP, remote device via a healthcare professional interface comprising a web-based portal and/or an app-based portal to allow a health care professional to access the collected physiological data of the patient on the RMS.

14. A computer program product comprising instructions, which, when executed by at least one processing unit, cause the at least one processing unit to perform the steps of the method according to claim 13.

15. A computer readable data carrier storing a computer program product according to claim 14.
